(19) United States
(12) Patent Application Publication
(10) Pub. No.: US 2019/0054123 A1
(43) Pub. Date: Feb. 21, 2019

(54) CELL SHEET COMPOSITION INCLUDING MESENCHYMAL STEM CELLS, AND METHOD FOR HEALING LUMINAL ORGAN USING SAME

(71) Applicant: TOKYO WOMEN'S MEDICAL UNIVERSITY, Tokyo (JP)

(72) Inventors: Nobuo Kanai, Tokyo (JP); Yasuhiro Maruya, Tokyo (JP); Kurodo Koshino, Tokyo (JP); Masanori Maeda, Tokyo (JP); Teruo Okano, Tokyo (JP)

(73) Assignee: TOKYO WOMEN'S MEDICAL UNIVERSITY, Tokyo (JP)

(21) Appl. No.: 16/072,109

(22) PCT Filed: Jan. 17, 2017
(86) PCT No.: PCT/JP2017/001432

§ 371 Date: Jul. 23, 2018

(30) Foreign Application Priority Data
Jan. 29, 2016 (JP) 2016-016497

(51) Int. Cl. A61K 35/28; A61L 27/38; A61P 1/00

(52) U.S. Cl. CPC A61K 35/28; A61L 27/3839; A61P 1/00; A61L 27/3834

(57) ABSTRACT

The present invention addresses the problem of providing a cell sheet composition for healing or preventing discharge from a wounded region of a luminal organ. The present invention also addresses the problem of providing a method in which the cell sheet composition is affixed to a wounded region of a luminal organ to heal or prevent discharge from the wounded region of the luminal organ. The present invention provides a cell sheet composition including mesenchymal stem cells which is characterized by being affixed to a wounded region of a luminal organ in order to heal or prevent discharge from the wounded region of the luminal organ. The present invention also provides a method in which the cell sheet composition including mesenchymal stem cells is affixed to a wounded region of a luminal organ to heal or prevent discharge from the wounded region of the luminal organ.

### FIELD

The present invention relates to a cell sheet composition containing mesenchymal stem cells, for healing or preventing leakage from a wound site of a luminal organ. The present invention further relates to a method in which a cell sheet composition containing mesenchymal stem cells is applied to the wound site of a luminal organ for healing or preventing leakage from the wound site of the luminal organ.

### BACKGROUND

When a malignant tumor arises in the gastrointestinal tract, the malignant tumor alone on the surface layer can be excised by Endoscopic Mucosal Resection (EMR) or Endoscopic Submucosal Dissection (ESD) using an endoscope, if detected in an early stage with minimal tissue invasion. For early stage cancer in the esophagus, for example, the lesion site can be excised by ESD using an endoscope. However, an ulcer forms at the site of excision after ESD, often causing stenosis of the esophagus. A method of applying an oral mucosal epithelial cell sheet has been developed for the purpose of preventing the occurrence of stenosis (NPL 1). An oral mucosal epithelial cell sheet is a type of regenerative medicine in which the oral mucosal epithelial cell sheet is obtained by harvesting a tissue sample from the oral mucosa of a patient, culturing the obtained oral mucosal epithelial cells on a temperature-sensitive culture dish, and culturing at a lower critical temperature, and it is transplanted at the resected surface of the esophageal inner wall following ESD. Stenosis of the esophagus is thus prevented.

With advanced cancer, however, the malignant tumor infiltrates deeply into tissue with the lesion becoming more extensive, and therefore tissue surrounding and including the lesion site must be excised, which requires the surgical procedures of suturing or anastomosis of the remaining gastrointestinal tract after excision of the lesion site. Gastrointestinal suture failure is a potential problem in such cases.

Gastrointestinal suture failure is a condition in which the anastomotic site fails to heal after anastomosis of the gastrointestinal tract and separates, and it is a complication following gastrointestinal surgery that can potentially result in leakage of intestinal contents into the thoracic cavity or abdomen, causing serious infectious disease and sometimes death. Suture failure occurs due to interference in the healing process (repair stage) of tissue at the anastomotic site, the causes of interference in the healing process during the repair stage including preoperative malnutrition, administration of drugs such as steroids, systemic factors due to chronic disease (diabetes, liver or kidney disorders), or local factors such as circulation disorders, hypertonia or infection around the anastomotic site. In order to prevent suture failure, various measures are taken such as making efforts to improve the general condition before surgery, development of automatic anastomosis devices, selecting an appropriate anastomosis method suited for the site and condition, and reducing tension and ensuring proper blood flow at the anastomotic site, but it is currently not possible to completely prevent suture failures.

One means for solving this problem has been reported in animal experiments using mesenchymal stem cells or adipose-derived stem cells, which have the effect of accelerating tissue wound healing, the majority of such transplant methods employing methods of injecting the cells by intravenous injection or local injection. The process of wound healing in a gastrointestinal anastomotic site is generally divided into an inflammatory stage, lasting up to 3-4 days after surgery, and a repair stage up to about 7 days after surgery. In the inflammatory stage up to 3-4 days after surgery, existing collagen is broken down by collagenase from inflammatory cells for reconstruction of the submucosal tissue at the anastomotic site, with fibroblasts subsequently proliferating in the repair stage, and collagen production increasing, to maintain continuous and physical tensile strength of the tissue up to 7 days after surgery (NPL 2). Therefore, in transplant methods involving injection of cells at the anastomotic site, not only is it difficult to hold the cells in their place, but the injected cells are also exposed to the protein-degrading enzyme collagenase during the inflammatory stage, whereby the cytotoxicity of collagenase can potentially interfere with the tissue repair function.

Moreover, although advances have been made with more powerful local and systemic treatments in recent years such as preoperative chemoradiotherapy and molecular-targeting therapy, even while they contribute to improving relapse and postoperative survival of gastrointestinal cancer, an increasing incidence of postoperative suture failure occurring with delayed wound healing has been a problem.

### CITATION LIST

### Non-Patent Literature

[NPL 1] Ohki T., et al. Treatment of oesophageal ulcerations using endoscopic transplantation of tissue-engineered autologous oral mucosal epithelial cell sheets in a canine model. Gut. 2006; 55(12):1704-1710.

[NPL 2] Hendriks T., Mastboom W J. Healing of experimental intestinal anastomoses. Parameters for repair. Dis Colon Rectum. 1990 October; 33(10):891-901.

### SUMMARY

### Technical Problem

Thus, while attempts have been made to develop technology for preventing and healing suture failure of luminal organs and especially the gastrointestinal tract, such technology has not yet been realized. It is an object of the present invention to provide a cell sheet composition for healing or preventing leakage from a wound site of a luminal organ. It is another object of the invention to provide a method in which a cell sheet composition is applied to the wound site of a luminal organ for healing or preventing leakage from the wound site of the luminal organ.

### Solution to Problem

The present inventors have conducted research and development based on examination of the problem from many angles, in order to solve the problems described above. As a result we have found, surprisingly, that when a cell sheet composition containing mesenchymal stem cells is applied to the wound site of a luminal organ, leakage from the wound site of the luminal organ is healed or prevented. Specifically, the present invention provides the following.

[1] A cell sheet composition containing mesenchymal stem cells, which is applied to a wound site of a luminal organ for healing or preventing leakage from the wound site of the luminal organ.

[2] The cell sheet composition according to [1], wherein the mesenchymal stem cells are mesenchymal stem cells derived from umbilical cord blood, placenta, bone marrow, adipose tissue, synovial membrane and/or pluripotent stem cells.

[3] The cell sheet composition according to [1] or [2], wherein the mesenchymal stem cells are adipose-derived stem cells.

[4] The cell sheet composition according to any one of [1] to [3], wherein the wound site is a sutured or anastomosed wound site.

[5] The cell sheet composition according to any one of [1] to [4], wherein the site of application is the outer wall of the luminal organ.

[6] The cell sheet composition according to any one of [1] to [5], wherein the luminal organ is the gastrointestinal tract.

[7] The cell sheet composition according to any one of [1] to [6], wherein the luminal organ is the intestinal tract.

[8] A method for healing or preventing leakage from the wound site of the luminal organ, comprising applying a cell sheet composition containing mesenchymal stem cells to a wound site of a luminal organ.

[9] The method according to [8], wherein the mesenchymal stem cells are mesenchymal stem cells derived from umbilical cord blood, placenta, bone marrow, adipose tissue, synovial membrane and/or pluripotent stem cells.

[10] The method according to [8] or [9], wherein the mesenchymal stem cells are adipose-derived stem cells.

[11] The method according to any one of [8] to [10], wherein the wound site is a sutured or anastomosed wound site.

[12] The method according to any one of [8] to [11], wherein the site of application is the outer wall of the luminal organ.

[13] The method according to any one of [8] to [12], wherein the luminal organ is the gastrointestinal tract.

[14] The method according to any one of [8] to [13], wherein the luminal organ is the intestinal tract.

### Advantageous Effects of Invention

The present invention provides a revolutionary effect for preventing or healing gastrointestinal suture failure occurring when the healing process (repair stage) of tissue in an anastomotic site is hindered by various factors, whereby the tissue repair function is maximized by the cell sheet composition of the invention and a method using it. This can potentially alleviate the significant reduction in patient QOL and social loss such as the input of medical resources, due to additional treatment such as artificial anus or intestinal fistula construction that may be required after suture failure.

### BRIEF DESCRIPTION OF DRAWINGS

FIG. 1 is a set of photographs showing the results of histological analysis of an adipose-derived stem cell sheet composition. The upper photograph shows the cell sheet composition. The middle photograph shows hematoxylin-eosin staining (HE staining) and the lower photograph shows vimentin staining, of a tissue section with a 4-layered sheet.

FIG. 2 is a set of graphs showing analysis of adipose-derived stem cell surface antigens.

FIG. 3 is a pair of photographs showing the differentiation-inducing ability of adipose-derived stem cells.

FIG. 4 is a photograph showing a colony-forming test for confirmation of the auto-replicating ability of adipose-derived stem cells.

FIG. 5 is a photograph showing graft survival after transplantation of an adipose-derived stem cell sheet composition.

FIG. 6A is a pair of photographs showing a small intestine anastomotic site.

FIG. 6B is a graph showing ulcer area in a small intestine anastomotic site.

FIG. 7A is a pair of photographs showing vascularization observed in a small intestine anastomotic site. Immunostaining was with anti-CD31 antibody.

FIG. 7B is a pair of photographs showing vascularization observed in a small intestine anastomotic site. Fluorescent immunostaining was with anti-CD31 antibody.

FIG. 7C is a graphic illustration of vascularization in a small intestine anastomotic site.

### DESCRIPTION OF EMBODIMENTS

For the purpose of the present invention, a luminal organ is an organ having a lumen structure, such as the gastrointestinal tract, vascular system, bladder and vagina, but it is preferably the gastrointestinal tract. For the present invention, the gastrointestinal tract is the organ running from the oral cavity to the anus, and it includes the esophagus, stomach, small intestine and large intestine, for example.

Throughout the present specification, the term “wound” means, loosely, a damaged state of a tissue or organ, and it includes burns, bedsores, bruises, incision wounds, brush burns, ulcers, operative wounds, gunshot wounds, blast wounds, puncture wounds, impalement wounds and bite wounds, for example. For the purpose of the present invention, the wound is preferably an incision wound, operative wound, puncture wound, impalement wound or bite wound in a luminal organ, and more preferably it is an operative wound. For the purpose of the present invention, an operative wound is a wound formed as a result of surgery, and for example, it is a wound formed using a scalpel, scissors, medical laser, forceps or snare, but it is not limited so long as it is a wound formed by an instrument normally used in surgery. For the purpose of the present invention, the term “wound site” refers to surrounding tissue that includes the site of the wound in a tissue or organ, and for example, it is within a radius of 10 cm, 8 cm, 5 cm, 3 cm or 2 cm from the site of the wound as the center. The extent and shape of the wound site is not limited as it will change depending on the shape of the wound.

For the purpose of the invention, the term “leakage” from a wound site in a luminal organ refers to the contents of the luminal organ leaking to the outside of the luminal organ, and when the luminal organ is the gastrointestinal tract, for example, it refers to leakage of solids such as food or its digested products, liquids such as gastric juice or saliva or gas such as air, out from the wound site into the thoracic cavity or intraperitoneal cavity. Particularly in the case of cancer of a luminal organ such as the gastrointestinal tract that has infiltrated deeply, it is necessary to resect part of the gastrointestinal tract. In such cases, the remaining gastrointestinal tract must be sutured or anastomosed. As mentioned above, suture failure may occur at the sutured or anastomosed portion of the gastrointestinal tract, for various reasons, resulting in leakage of contents. The present invention provides an effect of accelerating healing of a sutured or anastomosed wound site in a luminal organ and especially the gastrointestinal tract, to prevent or heal suture failure, by applying a cell sheet composition containing mesenchymal stem cells according to the invention to the wound site. By using the present invention it is also possible to exhibit significantly high compressive strength and to accelerate strength recovery at suturing or anastomotic sites of the gastrointestinal tract through which various substances pass, compared to when it is not used. This contributes to improved prognosis and quality of life (QOL) of patients after surgery.

According to the invention, the method of suturing or anastomosing the wound site of a luminal organ is not particularly restricted so long as it is a method used in conventional surgery. The suture thread used for suturing or anastomosing of the luminal organ may be either soluble or non-soluble, but a soluble suture thread is preferred from the viewpoint of invasiveness. The thickness of the suture thread may be selected as appropriate for the size and site of the wound.

The term “cell sheet composition” for the purpose of the invention means a cell group that is in the form of a single layer or multiple layers obtained by culturing on a cell culturing substrate and detaching from the cell culturing substrate. The method of obtaining the cell sheet composition may be, for example, a method in which cells are cultured on a stimuli-sensitive culture substrate coated with a polymer that changes its molecular structure by stimuli such as temperature, pH or light, and the stimuli-sensitive culture substrate surface is varied by changing the conditions of stimulation such as temperature, pH and light, to maintain the adhered state between the cells while detaching the cells from the stimuli-sensitive culture substrate in the form of a sheet, or a method in which cells are cultured on an arbitrary culture substrate and detached from the edges of the cell culturing substrate using physical forceps. A preferred mode is a method using a temperature-sensitive culture substrate as the stimuli-sensitive culture substrate, with the surface coated with a polymer that varies in hydration force in a temperature range of 0 to 80° C. In this method, cells are cultured on the temperature-sensitive culture substrate in a temperature range in which the hydration force of the polymer is weak, and the cells are subsequently cultured while changing the temperature of the culture solution to a temperature in which the hydration force of the polymer is strong, and detaching and recovering the cells in the form of a sheet. During this time, on a cell culturing substrate having the surface coated with a polymer that varies in hydration force within a temperature range of 0 to 80° C., the cells are cultured in a temperature range in which the hydration force of the polymer is weak. The temperature range will usually be a cell-culturing temperature, and is preferably 33° C. to 40° C., for example. The temperature-sensitive polymer used for the invention may be a homopolymer or a copolymer. Examples of such polymers include the polymers described in Japanese Unexamined Patent Publication HEI No. 2-211865.

A case using poly(N-isopropylacrylamide) as a stimuli-sensitive polymer, and specifically a temperature-sensitive polymer, will now be explained (temperature-sensitive culture dish). Poly(N-isopropylacrylamide) is known as a polymer having a lower critical solution temperature at 31° C., and when in the free state, dehydration takes place at a temperature of above 31° C. in water, resulting in aggregation of the polymer chains and opacity. Conversely, at temperatures of below 31° C., the polymer chains are hydrated and become dissolved in water. According to the invention, the polymer is coated and fixed onto the surface of a substrate such as a dish. Thus, while the polymer similarly undergoes dehydration on the culture substrate surface at a temperature of above 31° C., the culture substrate surface exhibits hydrophobicity because the polymer chains are immobilized on the culture substrate surface. Conversely, at a temperature below 31° C., the polymer on the culture substrate surface undergoes hydration, and since the polymer chains are coated on the culture substrate surface, the culture substrate surface exhibits hydrophilicity. The hydrophobic surface is a suitable surface allowing adhesion and proliferation of cells, while the hydrophilic surface is a surface that prevents adhesion of cells. Therefore, cooling the base to below 31° C. results in detachment of the cells from the substrate surface. If the cells are cultured to confluency on a culture surface, the cell sheet composition can be recovered by cooling the substrate to below 31° C. The temperature-sensitive culture dish is not particularly restricted so long as it has the same effect, and for example, it may be an Up Cell® marketed by CellSeed, Inc.

The animal source of the cells to be used for the invention may be a mammalian animal such as a human, rat, mouse, guinea pig, marmoset, rabbit, dog, cat, sheep, pig, goat, monkey, chimpanzee, or an immune-deficient animal of any of these species, or it may be a bird, reptile, amphibian, amphibian, fish, insect or the like. When the cell sheet composition of the invention is to be used for treatment of a human the cells are preferably human-derived, when it is to be used for treatment of a pig they are preferably pig-derived, when it is to be used for treatment of a monkey they are preferably monkey-derived, and when it is to be used for treatment of a chimpanzee they are preferably chimpanzee-derived. When a human is to be treated, they may be cells harvested from the patient (autologous cells), or cells harvested from another person (heterologous cells), or they may be a commercially available cell line.

Throughout the present specification, “mesenchymal stem cells” refers to undifferentiated cells that have the ability to differentiate into various types of mesenchymal cells such as adipocytes, chondrocytes, osteocytes, myoblasts, fibroblasts, stromal cells and/or tendon cells, and also having auto-replicating ability. According to the International Society for Cellular Therapy (ISCT), the following three minimal conditions are advocated for defining mesenchymal stem cells: (1) the capability of adhering to and being cultured on plastic under standard culturing conditions, (2) having the immunological feature of being positive for CD105, CD73 and CD90 and negative for CD45, CD34, CD14 or CD11b, CD79a or CD19 and HLA-DR, and (3) exhibiting differentiation potency to osteoblasts, adipocytes and chondroblasts in an in vitro differentiation system; however, the present invention is not limited to this definition. Also, CD29, CD44, CD106 and STRO-1 are additional positive markers for mesenchymal stem cells. According to the invention, the term “mesenchymal stem cells” is interpreted in as wide a sense as possible.

Mesenchymal stem cells are cells isolated from in vivo tissue such as bone marrow, adipose tissue, umbilical cord blood, dental pulp, synovial membrane or placenta, and they may be isolated using a known method.

For example, for bone marrow-derived mesenchymal stem cells, bone marrow fluid harvested from bone marrow, after separation of the hematocytes by density gradient centrifugation, is seeded in a plastic culture dish and cultured in an environment at 37° C., 5% CO₂, to allow isolation as adhering cells.

For adipose tissue-derived mesenchymal stem cells (adipose tissue-derived stem cells, or adipose-derived stem cells), harvested adipose tissue is minced and treated with collagenase type II at 37° C. for 1 hour for digestion, and medium is added prior to centrifugal separation. Next, the precipitated cells are rinsed with basal medium and filtered with a mesh such as a cell strainer, and then seeded on a plastic culture dish and cultured in an environment at 37° C., 5% CO₂, to allow isolation as adhering cells. Methods for isolating other tissue-derived mesenchymal stem cells are not limited so long as they are known methods.

The mesenchymal stem cells may also be mesenchymal stem cells obtained by inducing differentiation from pluripotent stem cells. Throughout the present specification, “pluripotent stem cells” refers to cells with auto-replicating ability and pluripotency, such cells having the ability to form different types of cells composing the body (being pluripotent). The term “auto-replicating ability” means the ability to produce two identical undifferentiated cells from a single cell. The pluripotent stem cells used for the invention include embryonic stem cells (ES cells), embryonic carcinoma cells (EC cells), trophoblast stem cells (TS cells), epiblast stem cells (EpiS cells), embryonic germ cells (EG cells), multipotent germline stem cells (mGS cells) and induced pluripotent stem cells (iPS cells). Mesenchymal stem cells induced from pluripotent stem cells using a known method (for example, Japanese Unexamined Patent Publication No. 2012-120486, or Fukuta M., et al., Derivation of mesenchymal stromal cells from pluripotent stem cells through a neural crest lineage using small molecule compounds with defined media. PLOS ONE, 2014; 9(12):e112291), may also be used.

The ability of mesenchymal stem cells to be induced to differentiate can be confirmed by a known method. For example, induced differentiation from mesenchymal stem cells to adipocytes can be confirmed by culturing in medium containing insulin and dexamethasone, and staining with Oil Red O. Induced differentiation from mesenchymal stem cells to osteocytes, for example, can be confirmed by adding ascorbic acid, β-glycerophosphoric acid and dexamethasone to the culture medium, carrying out culturing, and then staining with alkali phosphatase. Induced differentiation from mesenchymal stem cells to muscle can be confirmed by culturing in culture medium containing horse serum, and confirming appearance of fused cells specific to muscle cells. The induced differentiation from mesenchymal stem cells is not particularly restricted so long as it employs a known method. It is also sufficient to use a known method to confirm the presence or absence of induced differentiation, and for example, a gene or protein that is expressed only after differentiation has been induced may be detected by real-time PCR, or using a flow cytometer or the like.

The source of the mesenchymal stem cells to be used for the invention is not restricted, but it is preferably bone marrow or adipose tissue since their harvesting methods and separation methods are well established. An adipose tissue source will have a greater number of harvested mesenchymal stem cells than a bone marrow source, and is therefore preferred.

Depending on the source of the cells, the cells may have difficulty adhering onto the cell culturing substrate, and in such cases one or a mixture of two or more cell adhesion proteins such as collagen, laminin, laminin 5, fibronectin or Matrigel®, for example, may be precoated onto the cell culturing substrate before culturing of the cells. The method of coating such cell adhesion proteins may be based on a common method, an example being a method in which an aqueous solution containing the cell adhesion proteins is coated onto the cell culturing substrate surface, the aqueous solution is subsequently removed, and the substrate is rinsed.

According to the invention, the number of mesenchymal stem cells in the cell sheet composition is not restricted since it will depend on the size and extent of the wound onto which it is to be applied. The proportion of mesenchymal stem cells in the cell sheet composition of the invention is also not restricted, and it may be 30% or higher, 40% or higher, 50% or higher, 55% or higher, 60% or higher, 65% or higher, 70% or higher, 75% or higher, 80% or higher, 85% or higher, 90% or higher, 93% or higher, 95% or higher, 97% or higher, 98% or greater or 99% or higher, for example. A higher proportion of mesenchymal stem cells in the cell sheet composition will result in a greater effect of preventing or healing leakage at the wound site of the luminal organ.

The cells composing the cell sheet composition may include cells other than mesenchymal stem cells, and for example, vascular endothelial cells, vascular endothelial precursor cells, fibroblasts, epithelial cells or interstitial cells may be selected as appropriate for the site and purpose of transplantation. Cells derived from the tissue from which the mesenchymal stem cells were harvested may also be included.

According to the invention, the seeded cell count for preparation of the cell sheet composition will differ depending on the animal species and cell type, and it may be 0.3×10⁴ to 10×10⁶/cm², 0.5×10⁴ to 8×10⁶/cm², or 0.7×10⁴ to 5×10⁶/cm², for example. According to the invention, for recovery of the cell sheet composition by detachment from the temperature-responsive culture substrate, detachment can be achieved by adjusting the temperature of the culture substrate on which the cells are adhering in a confluent or subconfluent state, so that it is above the upper critical solution temperature or below the lower critical solution temperature of the coating polymer. The cell sheet composition may also be prepared in the culture solution, or in another isotonic solution, as appropriate for the purpose. In order to more rapidly detach and recover the cell sheet composition at high efficiency, a method of disturbing the culture substrate by gently tapping, a method of agitating the medium using a pipette, or a method of using forceps, may be employed, either alone or in combinations. The culturing conditions other than the temperature may be according to a common method. For example, the medium used may be medium containing known serum such as fetal bovine serum (FBS), or a serum-free medium may be used.

The form of the cell culturing substrate used to prepare the cell sheet composition to be used for the invention may be a dish, multiplate, flask or flat membrane form, for example. The material of the cell culturing substrate may be a compound that is commonly used for cell culturing, such as glass, modified glass, polystyrene, polymethyl methacrylate or polycarbonate, or a substance that generally imparts form, such as a polymer compound other than those mentioned above, or a ceramic.

The cell culturing substrate for preparation of the cell sheet composition to be used for the invention may be a cell culturing substrate comprising both regions where cells are adhering and regions where cells are not adhering, on the same culture surface, and for example, by using a cell culturing substrate comprising a plurality of circular cell-adhering regions and other regions where the cells are not adhering on the same culture surface, it is possible to prepare a plurality of cell sheets at one time. In this case, the shapes of the cell-adhering regions may be any desired shapes depending on the purpose, such as circular, square, triangular or rectangular, and their sizes may also be varied as appropriate. The method of forming the regions where cells are not adhering is not particularly restricted, and for example, it may be a method of coating with a hydrophilic polymer such as poly-N-acryloylmorpholine, polyacrylamide, polydimethylacrylamide, polyethylene glycol or cellulose, or a strongly hydrophobic polymer such as silicone polymer or fluorine polymer, as a non-cell-adhering polymer having low affinity with cells.

Since proteases such as Dispase or trypsin that are conventionally used when recovering adherent cells are not employed for the cell sheet composition of the invention, it produces virtually no damage to proteins expressed on the cell surfaces. Consequently, the lower surface of the cell sheet composition that has been detached from the cell culturing substrate (the surface on the side that was in contact with the cell culturing substrate) has an abundant amount of non-damaged adhesive proteins, and the cell-to-cell desmosome structure is maintained. Because it has such a structure, the cell sheet composition is suitable for application onto biological affected areas and for layering of the cell sheet composition. The protease Dispase is known to allow detachment of cells while maintaining 10 to 40% of the cell-to-cell desmosome structure, but it may simultaneously destroy basal membrane proteins present in the region between the cells and the culture substrate. In contrast, the cell sheet composition used for the invention allows detachment and recovery to be carried out in a state in which at least 60% of both the desmosome structure and basal membrane proteins remains, so that the different effects mentioned above can be obtained.

The cell sheet composition of the invention may also employ a layered cell sheet composition wherein a plurality of cell sheet compositions are layered. According to the invention, when a layered cell sheet composition is used, a greater number of cells are applied and the effect of preventing or healing leakage of a wound site in a luminal organ is further increased. The method for obtaining the layered cell sheet composition may be a method in which cell sheet compositions floating in culture solutions are drawn up from each culture solution using a pipette or the like, and then discharged onto a cell sheet composition on a different culture dish, and layered by a liquid medium flow, or a method in which layering is formed using a cell transferring tool. Among methods for producing a layered cell sheet composition of the invention, methods using a cell transferring tool are preferred to allow layering without damage to the cell sheet composition. A cell transferring tool need only have the function of allowing the cell sheet composition to be captured, and examples of materials to be used include polyvinylidene difluoride (PVDF), silicone resin, polyvinyl alcohol, urethane, cellulose and its derivatives, chitin, chitosan, collagen, gelatin and fibrin gel. The cell transferring tool used may be in the form of a stamp, membrane, porous membrane, nonwoven fabric or woven fabric, for example. According to an embodiment of the invention, the cell transferring tool is sufficient if it functions to recover the cell sheet composition without damage and to layer it onto a different cell sheet composition, and it is preferably a cultured cell-transferring tool having cell-adhering regions comprising one or more types of cell adhesion proteins, cell adhesion peptides or hydrophilic polymers. For example, Japanese Unexamined Patent Publication No. 2005-176812 discloses a cultured cell-transferring tool in the form of a stamp, having cell-adhering regions. The cultured cell-transferring tool in the form of a stamp prevents damage to the cell sheet composition by the cell-adhering regions, and allows recovery while preventing contraction of the cell sheet composition that occurs when the cell sheet composition is detached from the culture dish. The cell transferring tool can easily transfer a cell sheet composition onto a different cell sheet composition, while layering the cell sheet composition without shrinking. By layering cell sheet compositions without shrinking them it is possible to carry out layering between cell sheet compositions without formation of gaps between them, and to thus obtain a layered cell sheet composition having a high-density three-dimensional structure.

In the method for producing the cell sheet composition of the invention, culturing may also be carried out in medium containing added ascorbic acid (see Kato Y, et al. Allogeneic Transplantation of an Adipose-Derived Stem Cell Sheet Combined With Artificial Skin Accelerates Wound Healing in a Rat Wound Model of Type 2 Diabetes and Obesity. Diabetes. 2015 August; 64(8): 2723-34). A cell sheet composition containing mesenchymal stem cells that is obtained by culturing in medium containing added ascorbic acid, can be obtained as a tear-resistant cell sheet composition having higher strength than a cell sheet composition obtained by culturing without ascorbic acid. This allows a cell sheet composition to be obtained that is even more suitable for transplantation.

Factors that induce vascularization may also be added to the cell sheet composition. Examples of factors that induce vascularization include vascular endothelial growth factor (VEGF), fibroblast growth factor (FGF), angiopoietin, platelet-derived growth factor (PDGF), transforming growth factor-β (TGF-β), matrix metalloprotease (MMP), VE-cadherin, ephrin, plasminogen activator, inducible nitrogen monoxide synthase (iNOS), cyclooxygenase-2 (COX-2) and placental growth factor (PlGF). Including these in the cell sheet composition will further accelerate vascularization at the site of transplantation.

The site where the cell sheet composition of the invention is to be applied may be any wound site, but it is preferably a sutured or anastomosed wound site. If the site is sutured or anastomosed, tissues that have been inflamed by the wound will tightly bond, and application of the cell sheet composition of the invention at that site will increase the curative effect. Application of the cell sheet of the invention increases production of collagen at the wound site, accelerates reconstitution of the luminal organ surrounding the wound site, and accelerates healing of the wound site, to an extent that can also withstand pressure increase in the luminal interior. The site of application of the cell sheet composition of the invention may be the inner wall and/or outer wall of a luminal organ, or it may be only on the outer wall, from the viewpoint of easier application during surgery.

### EXAMPLES

The present invention will now be explained in greater detail by examples, with the understanding that the invention is not limited in any way by the examples. The experimental protocol using minipigs for the Examples was that approved by the Ethics Committee for Animal Experiments at Tokyo Women's Medical University, and it was conducted according to the “Guidelines for Management and Use of Experimental Animals” (1996 revised edition) published by the U.S. National Institutes of Health (NIH).

<Animals, Reagents and Kits Used>
Female minipigs (age 5-6 months, NIBS minipigs, Nippon Institute for Biological Science)
Penicillin/streptomycin (INVITROGEN, #15140122)
Fetal bovine serum (FBS; Japan Bio Serum, #73106-23R1501)
Trypsin-EDTA (×1) (Nacalai Tesque, Inc., #32777-44)
L-ascorbic acid phosphate magnesium salt n-hydrate (Wako Pure Chemical Industries, Ltd., #013-19641)
Povidone-iodine (Isojin^{R}, Meiji Seika Kaisha, Ltd., #50400)
Collagenase (Serva Corp., #17465 NB 4G Proved Grade)
Distilled water (Otsuka Pharmaceutical Co., Ltd.)
RNeasy®, Fibrous Tissue Mini Kit (Qiagen, #74704)
Mitomycin C (Wako Pure Chemical Industries, Ltd., #134-07911)
Hepatocyte Growth Factor (Hepapoietin A, Scatter Factor) (HGF) ELISA Kit (antibodies-online. Com, #ABIN367412)
FGF basic Pig ELISA Kit (Abcam, #ab156467)

<Antibodies Used>
Alexa Fluor® 647 Mouse Anti-Pig CD29 (BD Pharmingen, #561496)
Anti-CD44 antibody [IM7] (FITC) (Abcam, #ab19622)
APC Mouse Anti-Human CD90 (BD Pharmingen, #561971)
CD105 Ms mAb to CD105 (Abcam, #ab69772)
PE Mouse Anti-Rat CD31 (BD Pharmingen, #555027)
Monoclonal Antibody to CD45/LCA (CD45R)-PE (Acris Antibodies, Inc., #SM563R)
CD31 antibody; Anti-CD31 antibody (Abcam, #ab28364)
CD29 negative control: Alexa Fluor® Mouse IgG1, κ Isotype Control (BD Pharmingen, #557714)
CD44 negative control: Mouse IgG (FITC)-Isotype Control (Abcam, #ab37356)
CD90 negative control: APC Mouse IgG1, κ Isotype Control (BD Pharmingen, #555751)
CD105 negative control: Mouse IgG2a, κ Isotype (PE/Cy7) (Abcam, #ab103534)
CD105 secondary antibody: Gout pAb to Ms IgG2a PE/Cy7 (Abcam, #ab130787)
CD31 negative control: CD29 control (BD Pharmingen, BD557714)
CD45 negative control: PE Mouse IgG1, κ Isotype Control (BD Pharmingen, #550617)

<Equipment Used>
75 cm² flask (BD Falcon, #353810)
3.5 cm (35 mm) temperature-sensitive culture dish (UpCell®) (CellSeed, Inc., #CS3007)

<Devices Used>
Manometer PG-100B (Nidec Copal Corp., #PG-100B-102R-MX2T)
Flow cytometer (Gallios, Beckman Coulter)
FACS analysis software (Kalusa, Beckman Coulter)
Real-time PCR (Step One Plus™ Real-Time PCR System, Thermo Fisher Scientific, #4379216)

<Primers Used>

The primers used for real-time PCR in the Examples were purchased from Applied Biosystems. Information for the primers are as follows.
ACTB (β-actin);

Taq Man® Gene Expression Assays, β-actin

Assay ID: Ss03376081_ml
Collagen 1;

Taq Man® Gene Expression Assays, collagen, type I, alpha 1

Assay ID: Ss03373340_ml
Collagen 3;

Taq Man® Gene Expression Assays, collagen, type III, alpha 1

Assay ID: Ss04323790_ml

1. Experiment Method

1-1. Isolation and Culturing of Adipose-Derived Stem Cells, and Preparation of Cell Sheet Composition

### (1) Isolation of Adipose-Derived Stem Cells

The adipose-derived stem cells were isolated by the method described in an article by Watanabe N. et al. (Watanabe N., et al., Genetically Modified Adipose Tissue-Derived Stem/Stromal Cells, Using Simian Immunodeficiency Virus-Based Lentiviral Vectors, in the Treatment of Hemophilia B. Hum Gene Ther. 2013 March; 24(3): 283-294). Specifically, under local anesthesia, 20 g of subcutaneous fat of the abdominal wall of NIBS minipigs (6 months, 16-20 kg) was harvested while minimizing inclusion of blood cell components. The harvested adipose tissue was then sterilized and disinfected with povidone iodine, and rinsed twice with antibiotic-containing medium (1% penicillin/streptomycin-containing DMEM). After rinsing, tissue strips were thinly cut on a dish using a scissors. After placing 4 g of each into a 50 ml tube and adding 35 ml of antibiotic-containing medium, 1 ml of collagenase at a concentration of 0.27 pzu/ml was added to each. Shaking was carried out for 1 hour at 37° C., 150 rpm. Centrifugation was then performed for 5 minutes at 4° C., 300 G. The tube was manually shaken for 30 seconds. Centrifugation was again performed for 5 minutes at 4° C., 300 G. The large tissue portions floating on the tube surface were removed and passed through a 100 μm cell strainer (BD Japan Becton Dickinson, #352360). They were then passed through a 40 μm cell strainer (BD Japan Becton Dickinson, #352340). Centrifugation was performed for 5 minutes at 4° C., 1500 rpm. After removing the supernatant and suspending the pellet in medium containing 10% FBS, it was seeded on five 75 cm² flasks and culturing was carried out in an incubator at 37° C.

(2) Culturing of Adipose-Derived Stem Cells and Preparation of Cell Sheet Composition

On the 3rd day after cell seeding, the medium was exchanged, and on the 5th day the cells were detached with 0.25% trypsin and subcultured on ten 75 cm² flasks. Two to three days after subculturing, they were again subcultured. After another 2-3 days, the cells were again detached with 0.25% trypsin, and after measuring the cell count, 2.3×10⁶ cells were suspended in 2 ml of medium, seeded on 35 mm UP Cell^{R} and cultured for 2 days at 37° C. After 2 days, the medium was exchanged with medium containing 16.4 μg/ml ascorbic acid. After another 2 days, the medium was exchanged with medium containing ascorbic acid, and immediately before transplantation of the cell sheet composition, the cells were incubated for 20-30 minutes with an incubator at 20° C. and recovered as a sheet.

(3) Histological Analysis of Adipose-Derived Stem Cell Sheet Composition

The cell sheet composition recovered by the method described above was embedded with compound and then frozen with liquid nitrogen to prepare a tissue section. It was then subjected to hematoxylin-eosin staining and vimentin immunostaining with anti-vimentin antibody (Abcam, #ab8069).

(4) Confirming Cultured Cells as Adipose-Derived Stem Cells

It was confirmed that the cells cultured using the cells of the 3rd subculturing immediately before seeding on UP Cell® were adipose-derived stem cells. The phenotypes of the cells were confirmed with a flow cytometer, using stem cell markers (CD29-, CD44-, CD90- and CD105-positive, CD31- and CD45-negative). Separately, inducement of differentiation to fat and bone was confirmed by a known method to evaluate the function (pluripotency) of the stem cells (see Kato Y, et al. Allogeneic Transplantation of an Adipose-Derived Stem Cell Sheet Combined With Artificial Skin Accelerates Wound Healing in a Rat Wound Model of Type 2 Diabetes and Obesity. Diabetes. 2015 August; 64(8):2723-34). The auto-replicating ability was also evaluated by a known method, using a colony formation test (reference Kato Y, et al. Allogeneic Transplantation of an Adipose-Derived Stem Cell Sheet Combined With Artificial Skin Accelerates Wound Healing in a Rat Wound Model of Type 2 Diabetes and Obesity. Diabetes. 2015 August; 64(8):2723-34).

(5) Confirming Secretion of HGF and FGF2 from Prepared Adipose-Derived Stem Cell Sheet Composition

After 24 hours of culturing the adipose-derived stem cell sheet composition, the culture medium was taken and the HGF and FGF2 in the culture solution were measured using a Hepatocyte Growth Factor (Hepapoietin A, Scatter Factor) (HGF) ELISA Kit and an FGF basic Pig ELISA Kit, according to the procedural manuals included with the kits.

1-2. Transplantation of Adipose-Derived Stem Cell Sheet Composition

(1) Method of Transplantation Into Gastrointestinal Anastomotic Site

The adipose-derived stem cell sheet composition recovered as a sheet by the method described above was transplanted onto the serous membrane surface of an anastomotic site after anastomosis of a gastrointestinal tract, with the basal membrane surface of the cell sheet composition adhering. Three cell sheet compositions were transplanted so as to cover the entire periphery of the anastomotic site.

(2) Transplantation Into Delayed Wound Healing Model of Minipig Small Intestine Suture

After locally injecting 2 ml of 100 μg/ml mitomycin C under the serous membrane of minipig small intestine, and ligation and separation of six blood vessels at the projected suture site, 2 cm on the mesenteric-facing side was incised, and then the site was sutured by Gambee suture using 4-0 Vicryl, with 5 needle penetrations to prepare a delayed wound healing model of a small intestine suture. After which the adipose-derived stem cell sheet composition was transplanted at the site, the wound healing accelerating effect and reinforcing effect at the sutured section by transplantation of the adipose-derived stem cell sheet composition were verified. Eight small intestine sutured sections were prepared for each minipig, and bypasses were created by side-to-side anastomosis on the adoral end and the anal end of the 8 sutured sections. Just before transplantation of the cell sheet compositions, random selection was made of 4 sites as cell sheet composition transplanted groups and 4 sites as cell sheet composition non-transplanted groups, and in each of the cell sheet composition transplanted groups, 3 adipose-derived stem cell sheet compositions were transplanted so that each sutured section was covered. An antibiotic was administered by drip infusion on the day of surgery and the following day, meals were provided from the 3rd day after surgery, and laparotomy was again performed on the 7th day after surgery. After extracting the sutured intestinal tracts, the animals were sacrificed by intravenous injection of potassium chloride.

(3) Examination of Graft Survival After Transplantation of Adipose-Derived Stem Cell Sheet Composition

For evaluation of the post-transplant engraftment, a fluorescent dye PKH26GL (Sigma, Product No.: PKH26GL-1KT) was added to the cell sheet composition immediately before transplantation. Each anastomotic site extracted on the 7th day after transplantation was embedded in an embedding agent for frozen tissue section preparation (product name “O.C.T. Compound” by Sakura Finetek Japan Co., Ltd., #4583), and was then frozen with liquid nitrogen to prepare a tissue section. The state of engraftment of the adipose-derived stem cell sheet composition was then confirmed with a fluorescent microscope.

(4) Examination of Physical Strength of Small Intestine Sutured Section (Pressure Test)

The small intestine sutured section was subjected to a pressure test with reference to published literature (Ikeda T., et al., Evaluation of techniques to prevent colorectal anastomotic leakage. J Surg Res. 2015 April; 194(2):450-7). Specifically, accretion of the sutured section in the extracted intestinal tract was gently detached, and a 2 cm portion was separated and disengaged from each sutured section. An extension tube (5C13M by Top Co.) was inserted at the adoral end of the sutured section, and ligated with #2 silk thread (Japanese Industrial Standards JIS-T4101), while the anal end of the sutured section was gripped at the stump with Lister intestinal forceps. The extension tube was then connected with a three-way stopcock drip tube, and in turn connected to a pressure-measuring manometer. The anastomotic site was submerged in 1500 ml of physiological saline, a 50 ml syringe was connected to the three-way stopcock, air was injected, and the pressure at the first moment of leakage of air from the sutured section was measured.

(5) Visual Evaluation of Sutured Section Area

After extraction of the pig sutured sections that had not been subjected to pressure testing, an incision was made on the mesenteric side, and each sample was spread out and fixed on the mucosal surface and photographed with a digital camera. The proportions of ulcerated area in regions of within 1 cm from the adoral ends and the anal ends of the sutured sections seen in the photographs were calculated using image analysis software (Image J (Ver.1.48)), and compared.

(6) Histological Evaluation

On the 7th day after surgery, the extracted samples were embedded in compound, and then frozen with liquid nitrogen to prepare tissue sections. Hematoxylin-eosin staining, Sirius Red staining and CD31 immunostaining were performed. For CD31, the positive cell density in each visual field was measured. The average value for the proportion of CD31-positive cells in 3 visual fields for each specimen was calculated, and the average value for each group was determined.

(7) Molecular Biological Analysis

On the 7th day after surgery, RNA was extracted from the sutured sections of the extracted samples. Synthesis of cDNA from the RNA was carried out by reverse transcription reaction. The transcription amounts of type I collagen and type III collagen were quantified by real-time PCR, and divided by the amount of β-actin transcription as an endogenous control.

(8) Statistical Analysis

The data were expressed as mean±SE. Comparison between the groups was conducted by t-test, with a P value of less than 0.05 considered as significant.

2. Results

(1) Histological Analysis of Adipose-Derived Stem Cell Sheet Composition

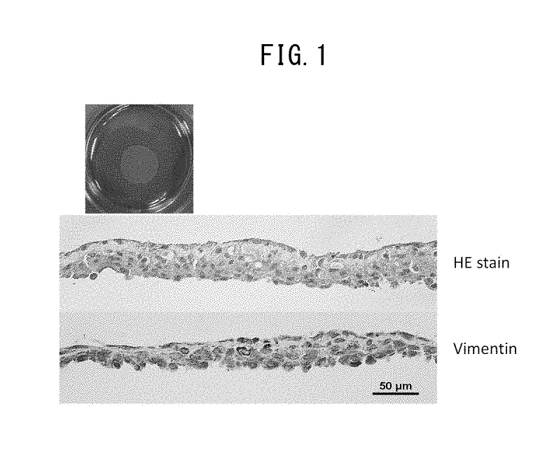

Four-layer sheets with diameters of 1.3 cm were histologically examined. The results were vimentin-positive in immunostaining, which was consistent with an adipose-derived stem cell sheet composition. (FIG. 1)

(2) Confirming Cultured Cells as Adipose-Derived Stem Cells

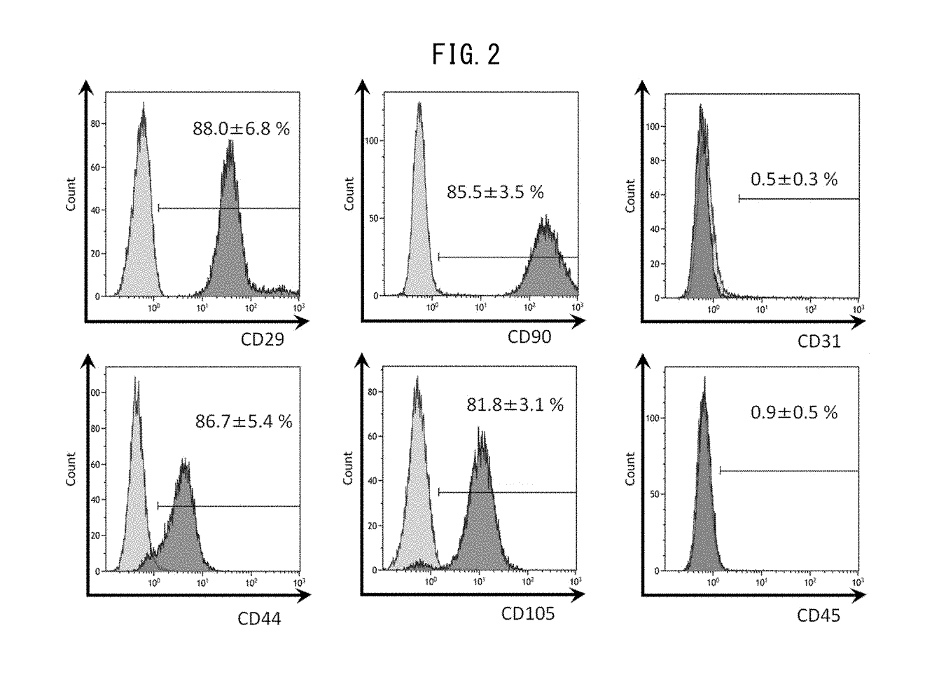

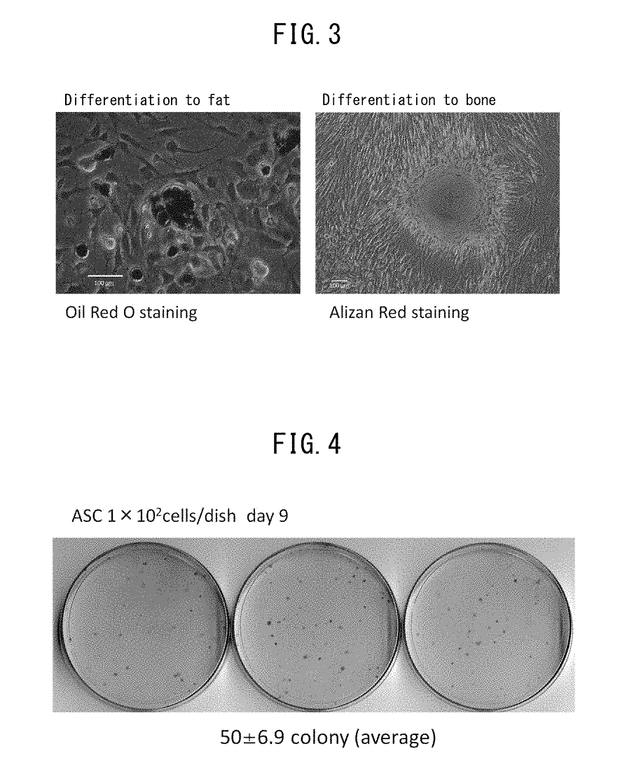

Expression of surface antigens was confirmed with a flow cytometer, to determine CD29-, CD44-, CD90- and CD105-positivity and CD31- and CD45-negativity. In addition, differentiation to fat and bone was confirmed in a differentiation-inducing test, and auto-replicating ability was confirmed in a colony formation test. (FIGS. 2 to 4)

(3) Secretion of HGF and FGF2 From Prepared Adipose-Derived Stem Cell Sheet Composition

HGF and FGF2 were detected by ELISA in culture solution obtained by culturing the adipose-derived stem cell sheet composition for 24 hours (FGF: 106.9 pg/ml, HGF: 1.38 ng/ml).

(4) Examination of Graft Survival After Transplantation of Adipose-Derived Stem Cell Sheet Composition

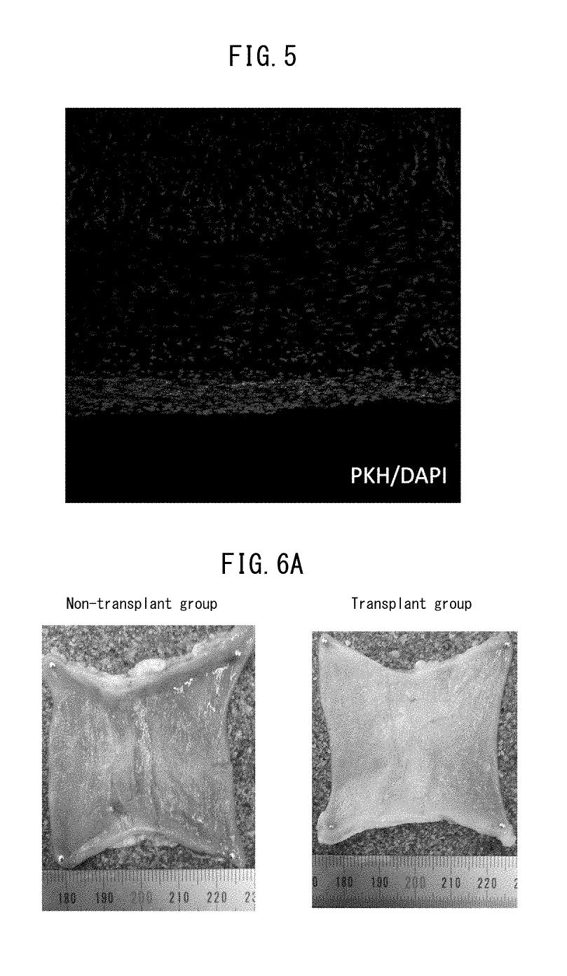

On the 7th day after transplantation, residue of PKH26GL in a sheet form on the serous membrane surface of the extracted anastomotic site was confirmed with a fluorescent microscope, confirming that the adipose-derived stem cell sheet composition had engrafted onto the serous membrane surface of the anastomotic site. (FIG. 5)

(5) Examination of Physical Strength of Small Intestine Sutured Section (Pressure Test)

The results of the pressure test at the anastomotic site on the 7th day after surgery were 211.3±25.4 mmHg in the non-transplant group and 276.0±29.6 mmHg in the adipose-derived stem cell sheet composition-transplant group, indicating significantly higher pressure resistance in the adipose-derived stem cell sheet composition-transplant group (n=4, p<0.05).

(6) Visual Evaluation of Anastomotic Site (Ulcerated Area)

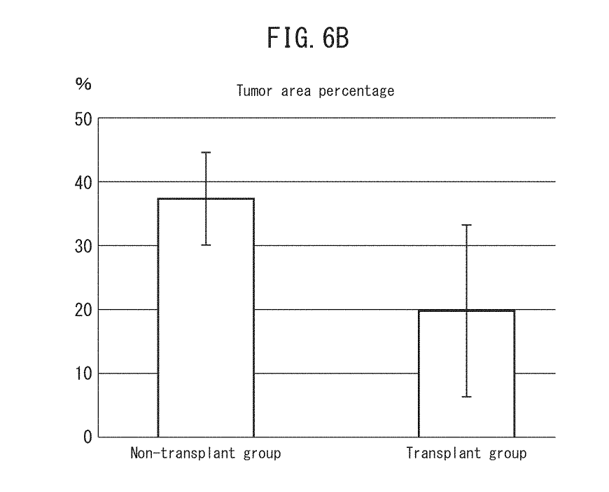

The proportion of ulcerated area on the mucosal surface surrounding the sutured section on the 7th day after surgery was 37.3±7.2% in the non-transplant group and 19.8±13.4% in the transplant group (n=4, p<0.05). (FIG. 6A and FIG. 6B)

(7) Histological Evaluation

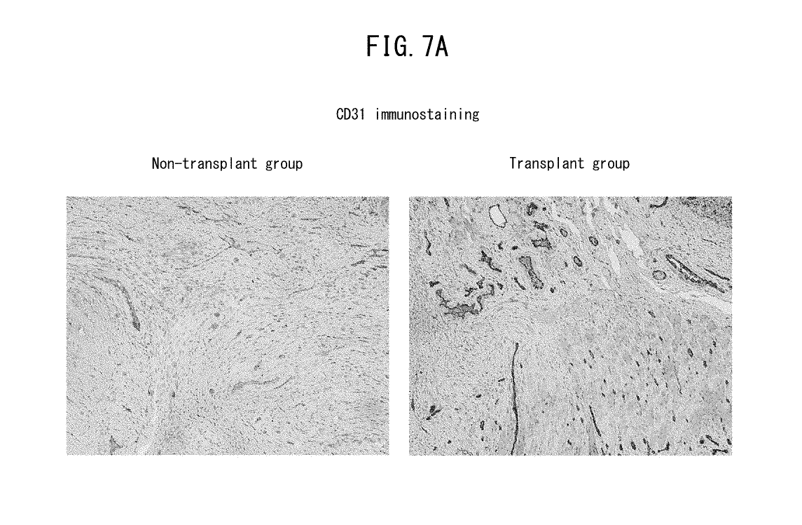

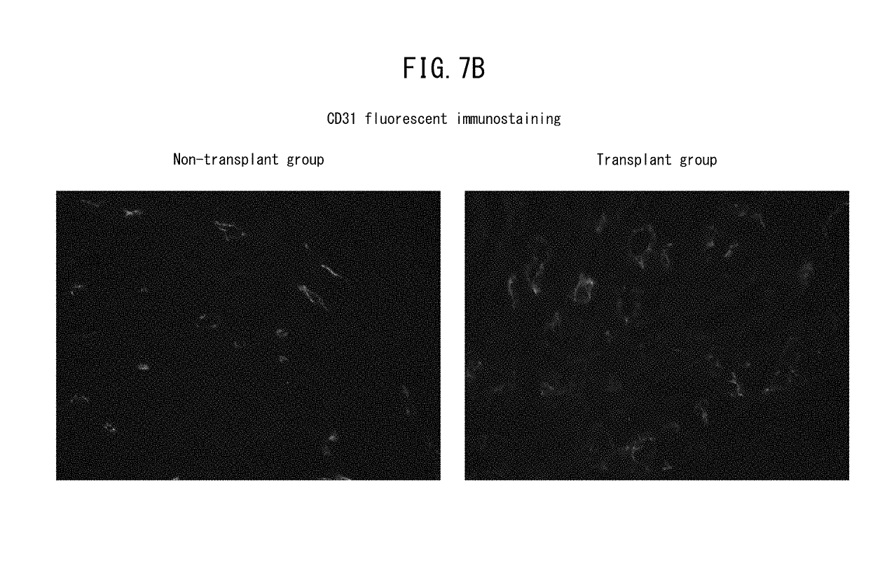

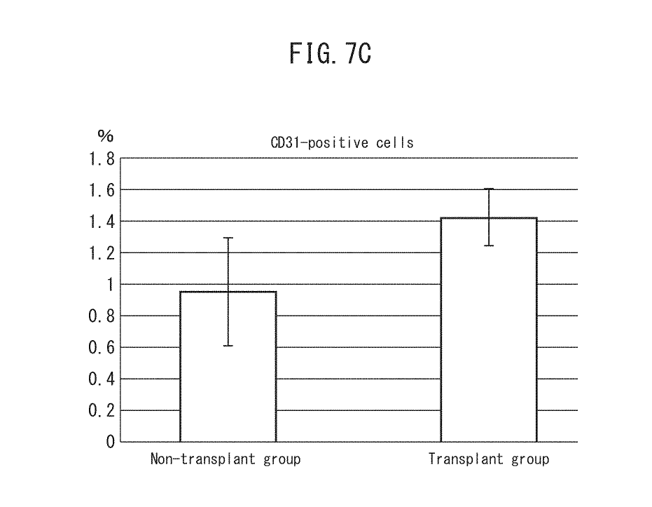

As a result of CD31 immunostaining, the proportion of CD31-positive cells per visual field was 0.95±0.33% in the non-transplant group and 1.42±0.17% in the transplant group, indicating that the adipose-derived stem cell sheet composition-transplant group had significantly accelerated vascularization compared to the non-transplant group (n=4, p<0.05). (FIGS. 7A to C)

(8) Molecular Biological Examination

Expression of type I collagen was 0.64±0.32 in the non-transplant group and 1.10±0.40 in the transplant group (n=4, p>0.05). Expression of type III collagen was 0.45±0.08 in the non-transplant group and 0.69±0.21 in the transplant group, indicating significantly higher expression in the adipose-derived stem cell sheet composition-transplant group (n=4, p<0.05). This suggests that transplantation of an adipose-derived stem cell sheet composition onto the serous membrane surface of a delayed wound healing model of a small intestine suture accelerates wound healing at the sutured section.

1. A cell sheet composition containing mesenchymal stem cells, which is applied to a wound site of a luminal organ for healing or preventing leakage from the wound site of the luminal organ.

2. The cell sheet composition according to claim 1, wherein the mesenchymal stem cells are mesenchymal stem cells derived from umbilical cord blood, placenta, bone marrow, adipose tissue, synovial membrane and/or pluripotent stem cells.

3. The cell sheet composition according to claim 1, wherein the mesenchymal stem cells are adipose-derived stem cells.

4. The cell sheet composition according to claim 1, wherein the wound site is a sutured or anastomosed wound site.

5. The cell sheet composition according to claim 1, wherein the site of application is the outer wall of the luminal organ.

6. The cell sheet composition according to claim 1, wherein the luminal organ is the gastrointestinal tract.

7. The cell sheet composition according to claim 1, wherein the luminal organ is the intestinal tract.

8. A method for healing or preventing leakage from the wound site of the luminal organ, comprising applying a cell sheet composition containing mesenchymal stem cells, to a wound site of a luminal organ.

9. The method according to claim 8, wherein the mesenchymal stem cells are mesenchymal stem cells derived from umbilical cord blood, placenta, bone marrow, adipose tissue, synovial membrane and/or pluripotent stem cells.

10. The method according to claim 8, wherein the mesenchymal stem cells are adipose-derived stem cells.

11. The method according to claim 8, wherein the wound site is a sutured or anastomosed wound site.

12. The method according to claim 8, wherein the site of application is the outer wall of the luminal organ.

13. The method according to claim 8, wherein the luminal organ is the gastrointestinal tract.

14. The method according to claim 8, wherein the luminal organ is the intestinal tract.
